(19) United States
(12) Patent Application Publication
(10) Pub. No.: US 2008/0076720 A1
(43) Pub. Date: Mar. 27, 2008

(54) Personal care compositions having kinetin or zeatin

(76) Inventors: Sancai Xie, West Chester, OH (US); Kotikanyadanam Sreekrishna, Mason, OH (US); Abby Ballard Newland, Lawrenceburg, IN (US); Charles Carson Bascom, Hamilton, OH (US); Joseph Robert Kaczvinsky JR., Cincinnati, OH (US); Karen Marie Lammers, North Bend, OH (US); Kristina Emma Inge Vanoosthuyze, Horsell Woking (GB); Donald Lynn Bissett, Hamilton, OH (US); Larry Richard Robinson, Loveland, OH (US); Rosemarie Osborne, Oxford, OH (US); Paul Robert Tanner, Lebanon, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/804,802

(22) Filed: May 21, 2007

Related U.S. Application Data
(63) Continuation-in-part of application No. 11/482,314, filed on Jul. 7, 2006.
(63) Continuation-in-part of application No. 10/627,533, filed on Jul. 25, 2003.
(63) Continuation-in-part of application No. 10/977,848, filed on Oct. 29, 2004.
(63) Continuation-in-part of application No. 10/780,267, filed on Feb. 17, 2004.
Application No. 10/780,267 is a continuation of application No. 10/379,252, filed on Mar. 4, 2003, now abandoned.
(60) Provisional application No. 60/697,819, filed on Jul. 8, 2005.
(60) Provisional application No. 60/516,502, filed on Oct. 31, 2003.

(51) Int. Cl. A61K 31/52; A01N 1/02; A61K 31/7008; A61K 38/02; A61K 38/06; A61K 38/08

(52) U.S. Cl. 514/17; 435/1.1; 514/18; 514/2; 514/263.23; 514/263.4; 514/62

(57) ABSTRACT

A personal care composition may comprise a member selected from a group consisting of kinetin, zeatin, and combinations thereof; niacinamide; and a dermatologically-acceptable carrier. A personal care composition may comprise an N-acyl amino acid compound, a skin care active, a member selected from a group consisting of kinetin, zeatin, and combinations thereof, and a dermatologically-acceptable carrier. A personal care composition may comprise a hexamidine compound, a skin care active, a member selected from a group consisting of kinetin, zeatin, and combinations thereof, and a dermatologically acceptable carrier.

### CROSS REFERENCE TO RELATED APPLICATION

This application is a continuation-in-part of U.S. application Ser. No. 11/482,314 filed Jul. 7, 2006, which claims the benefit of U.S. Provisional Application No. 60/697,819, filed Jul. 8, 2005; this application is a continuation-in-part of U.S. application Ser. No. 10/627,533 filed Jul. 25, 2003; this application is a continuation-in-part of U.S. application Ser. No. 10/977,848, which claims the benefit of U.S. Provisional Application No. 60/516,502, filed Oct. 31, 2003; and this application is a continuation-in-part of U.S. application Ser. No. 10/780,267 filed Feb. 17, 2004, which is a continuation of Ser. No. 10/379,252 filed Mar. 4, 2003. The aforementioned applications are hereby incorporated by reference.

### FIELD OF THE INVENTION

The present invention relates to personal care compositions comprising kinetin and/or zeatin and other skin care actives.

### BACKGROUND OF THE INVENTION

A number of personal care products currently are available to consumers, which are directed toward improving dry skin. The outermost layer of the skin, the stratum corneum, receives water by being brought into direct contact with water or via diffusion from the underlying epidermis. The diffusion process is controlled by the water content of the skin as well as the concentration gradient. In a very dry environment, evaporative water loss from the stratum corneum can be significant and often exceeds the rate of replacement by diffusion. Compositions containing humectants, occlusive or semi-occlusive substances, and/or materials that improve barrier function can inhibit or retard evaporative water loss, but have the disadvantage of only minimally affecting diffusion.

Aquaporins are a class of membrane proteins within mammalian skin that regulate the transport of water, glycerol, and other solutes across the plasma membrane. Without being limited by theory, two major aquaporin membrane proteins, AQP-3 and AQP-9, are expressed in skin. AQP-3 is a transporter protein in the plasma membrane of keratinocytes, which transports water and glycerol into the vascular-free epidermis from the dermis. When AQP-3 gene is inactivated, multiple symptoms of damaged skin, such as lower water content, leaky skin barrier, delayed wound healing and impaired skin elasticity, are observed. It is believed that an increase in the expression of AQP-3 in skin improves skin hydration, thus minimizing the visual signs of dry or photo-damaged skin, and delivering benefits in skin moisturization, appearance, tone, texture, and firmness. There exists a need to provide a personal care composition that stimulates aquaporin membrane proteins.

However, many consumers are concerned not only with dry-skin but also with the health and physical appearance of their skin. Consumers are often very concerned about the signs of skin aging such as deep wrinkles, fine lines, skin lines, crevices, bumps, large pores, unevenness or roughness, loss of skin elasticity, discoloration, and the like. There exists a need for a personal care composition that not only stimulates aquaporin membrane proteins but also contains components capable of regulating and/or improving skin condition which includes the signs of skin aging.

### SUMMARY OF THE INVENTION

The present invention relates to personal care composition addressing the aforementioned need. The present invention further relates to methods of using the personal care compositions.

The following represent some non-limiting embodiments of the present invention.

In one embodiment, a personal care composition is disclosed comprising a member selected from a group consisting of kinetin, zeatin, and combinations thereof; niacinamide; and a dermatologically-acceptable carrier.

In another embodiment, a personal care composition is disclosed comprising an N-acyl amino acid compound selected from the group consisting of N-acyl amino acid, its isomers, its salts, and derivatives thereof; a skin care active selected from the group consisting of sugar amine, vitamin B₃, retinoids, peptides, phytosterol, dialkanoyl hydroxyproline, hexamidine, salicylic acid and their derivatives, and combinations thereof; a member selected from a group consisting of kinetin, zeatin, and combinations thereof; and a dermatologically-acceptable carrier.

In another embodiment, a personal care composition is disclosed comprising a hexamidine compound selected from the group consisting of hexamidine, its salts, and its derivatives; a skin care active selected from the group consisting of sugar amines, vitamin B₃, retinoids, peptides, phytosterols, their derivatives, and combinations thereof; a member selected from a group consisting of kinetin, zeatin, and combinations thereof; and a dermatologically acceptable carrier.

In other embodiments, methods of regulating the condition of mammalian keratinous tissue using the personal care composition are disclosed.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

Herein, “personal care composition” means compositions suitable for topical application on mammalian keratinous tissue. “Skin care actives,” or “actives,” as used herein, means compounds that, when applied to the skin, provide a benefit or improvement to the skin. It is to be understood that skin care actives are useful not only for application to skin, but also to hair, nails, and other mammalian keratinous tissue.

“Keratinous tissue,” as used herein, refers to keratin-containing layers disposed as the outermost protective covering of mammals which includes, but is not limited to, skin, hair, nails, cuticles, etc.

“Topical application”, as used herein, means to apply or spread the compositions of the present invention onto the surface of the keratinous tissue.

“Dermatologically acceptable,” as used herein, means that the compositions or components described are suitable for use in contact with human keratinous tissue without undue toxicity, incompatibility, instability, allergic response, and the like.

“Effective amount” as used herein means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

Herein, “delivery enhancement device” means any device that increases the amount of active ingredient applied to and/or into the skin relative to the amount of active ingredient that is delivered without using the device.

Herein, “regulating skin condition” means improving skin appearance and/or feel, for example, by providing a smoother appearance and/or feel. Herein, “improving skin condition” means effecting a visually and/or tactilely perceptible positive change in skin appearance and feel. Conditions that may be regulated and/or improved include, but are not limited to, one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, and graying hair.

As used herein, “signs of skin aging,” include, but are not limited to, all outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

I. Kinetin and Zeatin

The composition of the present invention may comprise kinetin, zeatin, or combinations thereof. The composition of the present invention may comprise may from about 0.05% to about 5%, alternatively from about 0.1% to about 3%, and alternatively from about 0.1% to about 1.5%, of kinetin, zeatin, or combinations thereof. Without wishing to be bound by theory, it is believed that kinetin and zeatin stimulate the aquaporin membrane protein in the epidermis can provide benefits to the mammalian skin to which they are applied including but not limited to: enhanced barrier function, improved skin and hair hydration; increased firmness of skin; and reduction of fine lines and wrinkles.

It is further believed that the use of glycerin in conjunction with kinetin and/or zeatin results in enhanced beautification benefits to the mammalian skin to which it is applied by enhancing production of aquaporins in the epidermis (and the benefits thereof), improving skin hydration; improving barrier function and skin firming; increasing stratum corneum hydration; and reducing visible flakes, lines and wrinkles. In certain embodiments of the present invention, the composition may comprise from about 9% to about 30%, alternatively from about 10% to about 25%, alternatively from about 10% to about 20%, and alternatively from about 5% to about 9% of glycerin.

II. Skin Care Actives

The present invention may include additional hair and/or skin care actives, collectively referred to as “skin care actives,” selected from the group consisting of sugar amines, vitamin B₃, retinoids, peptides, dialkanoyl hydroxyproline, hexamidine, salicylic acid, phytosterol, sunscreen actives, water soluble vitamins, oil-soluble vitamins, their derivatives, their precursors, and combinations thereof.

1. Sugar Amines (Amino Sugars)—The composition of the present invention may comprise a sugar amine, which are also known as amino sugars. The sugar amine compounds useful in the present invention are described in PCT Publication WO 02/076423 and U.S. Pat. No. 6,159,485.

In one embodiment, the composition may contain from about 0.01% to about 15%, alternatively from about 0.1% to about 10%, and alternatively from about 0.5% to about 5% by weight of the composition, of the sugar amine.

Sugar amines can be synthetic or natural in origin and can be used as pure compounds or mixtures of compounds (e.g., extracts from natural sources or mixtures of synthetic materials). Glucosamine is generally found in many shellfish and can also be derived from fungal sources. As used herein, “sugar amine” includes isomers and tautomers of such and its salts (e.g., HCl salt) and is commercially available from Sigma Chemical Co.

Non-limiting examples of sugar amines useful herein include glucosamine, N-acetyl glucosamine, mannosamine, N-acetyl mannosamine, galactosamine, N-acetyl galactosamine, their isomers, salts (e.g., HCl salt) and derivatives. In one embodiment, the sugar amine is glucosamine, alternatively D-glucosamine and alternatively N-acetyl-D-glucosamine.

2. Vitamin B₃—The composition of the present invention may comprise a vitamin B₃ compound. Vitamin B₃ compounds are particularly useful for regulating skin condition as described in U.S. Pat. No. 5,939,082. In one embodiment, the composition may comprise from about 0.01% to about 50%, alternatively from about 0.1% to about 20%, alternatively from about 0.5% to about 10%, alternatively from about 1% to about 7%, and alternatively from about 2% to about 5%, of the vitamin B₃ compound. Non-limiting examples of vitamin B₃ compound include niacinamide, nicotinic acid, and nicotinyl alcohol. Non-limiting examples of derivatives of the vitamin B₃ compounds include nicotinic acid esters, including non-vasodilating esters of nicotinic acid (e.g., tocopheryl nicotinate, myristyl nicotinate).

In one embodiment, the composition may comprise niacinamide. Without wishing to be bound by theory, it is believed that the use of niacinamide in conjunction with kinetin and/or zeatin provides enhanced beautification benefits to mammalian skin by stimulating transport of water or glycerol within the epidermis, resulting in increased skin hydration, enhanced production of water binding molecules (e.g., hyaluronic acid) in skin, improved epidermal stratification and barrier formation; skin firming; and reduction in visible flakes, lines and wrinkles. In one embodiment of the present invention, the composition may comprise from about 1% to about 8%, alternatively from about 2% to about 5%, and alternatively from about 3% to about 5%, of niacinamide.

3. Retinoids—The composition of the present invention may comprise a retinoid, such that the resultant composition is suitable for regulating visible and/or tactile discontinuities in skin, for example, for regulating signs of skin aging. In one embodiment, the composition may comprise from about 0.001% to about 10%, alternatively from about 0.005% to about 2%, alternatively from about 0.01% to about 1%, and alternatively from about 0.01% to about 0.5%, by weight of the composition, of the retinoid. The optimum concentration used in a composition will depend on the specific retinoid selected since their potency may vary considerably.

As used herein, “retinoid” includes all natural and/or synthetic analogs of Vitamin A or retinol-like compounds which possess the biological activity of Vitamin A in the skin as well as the geometric isomers and stereoisomers of these compounds. The retinoid may be selected from retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), or mixtures thereof. In one embodiment, the retinoid is one other than retinoic acid. In one embodiment, the retinoid is selected from the group consisting of retinol, retinyl palmitate, retinyl acetate, retinyl propionate, retinal and combinations thereof. Alternatively, the retinoid is retinyl propionate, present in an amount of from about 0.1% to about 0.3%.

4. Peptides—The composition of the present invention may comprise a peptide, including but not limited to, di-, tri-, tetra-, penta-, and hexa-peptides and derivatives thereof. The composition may comprise from about 1×10⁻⁶% to about 20%, alternatively from about 1×10⁻⁶% to about 10%, and alternatively from about 1×10⁻⁵% to about 5%, and alternatively from about 0.001% to about 1%.

As used herein, “peptide” refers to peptides containing ten or fewer amino acids and their derivatives, isomers, and complexes with other species such as metal ions, including but not limited to copper, zinc, manganese, magnesium, etc. As used herein, peptide refers to both naturally occurring and synthesized peptides. Also useful herein are naturally occurring and commercially available compositions that contain peptides. for example, peptides derived from soy proteins, palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, and Cu-histidine-glycine-glycine (Cu-HGG, also known as IAMIN®). One example of a commercially available tripeptide derivative-containing composition is Biopeptide CL®, which contains 100 ppm of palmitoyl-gly-his-lys and is commercially available from Sederma.

5. Phytosterols—The compositions of the present invention may comprise one or more phytosterols selected from the group consisting of β-sitosterol, campesterol, brassicasterol, Δ5-avennasterol, lupenol, α-spinasterol, stigmasterol, their derivatives, analogs, and combinations thereof. In one embodiment, the phytosterol is selected from the group consisting of β-sitosterol, campesterol, brassicasterol, stigmasterol, their derivatives, and combinations thereof. In one embodiment, the phytosterol is stigmasterol.

Phytosterols can be synthetic or natural in origin and can be used as essentially pure compounds or mixtures of compounds (e.g., extracts from natural sources). Phytosterols are generally found in the unsaponifiable portion of vegetable oils and fats and are available as free sterols, acetylated derivatives, sterol esters, ethoxylated or glycosidic derivatives. In one embodiment, the phytosterols are free sterols. As used herein, “phytosterol” includes isomers, derivatives and tautomers of such and are commercially available from Aldrich Chemical Company, Sigma Chemical Company, and Cognis.

The composition of the present invention may comprise from about 0.0001% to about 25%, alternatively from about 0.001% to about 15%, alternatively from about 0.01% to about 10%, alternatively from about 0.1% to about 5%, and alternatively from about 0.2% to about 2% of the phytosterol.

6. Hexamidine—The composition of the present invention may comprise hexamidine. “Hexamidine,” as used herein, includes isomers, tautomers, salts and derivatives of hexamidine, including but not limited to organic acids and mineral acids, for example sulfonic acid, carboxylic acid, etc. A technical name for the hexamidine of the present invention is 4,4′-(hexamethylenedioxy)dibenzenecarboximidamide. Dermatologically acceptable salts include alkali metal salts, such as sodium and potassium; alkaline earth metal salts, such as calcium and magnesium; non-toxic heavy metal salts; and ammonium and trialkylammonium salts such as trimethylammonium and triethylammonium. Alternatively, the hexamidine is hexamidine isethionate, which is commercially available under the tradename ELASTAB® HP100 from Laboratoires Serobiologiques (Pulnoy, France).

The composition of the present invention may comprise from about 0.0001% to about 25%, alternatively from about 0.001% to about 10%, alternatively from about 0.01% to about 5%, and alternatively from about 0.02% to about 2.5% by weight of the composition.

7. Dialkanoyl Hydroxyproline Compounds—The compositions of the present invention may comprise one or more dialkanoyl hydroxyproline compounds and salts and derivatives thereof. The composition may comprise from about 0.01% to about 10%, alternatively from about 0.01% to about 5%, alternatively from about 0.1% to about 2% of a dialkanoyl hydroxyproline compound.

Suitable derivatives include but are not limited to esters, for example fatty esters, including, but not limited to tripalmitoyl hydroxyproline and dipalmityl acetyl hydroxyproline. A particularly useful compound is dipalmitoyl hydroxyproline. As used herein, dipalmitoyl hydroxyproline includes any isomers and tautomers of such and is commercially available under the tradename Sepilift DPHP® from Seppic, Inc. Further discussion of dipalmitoyl hydroxyproline appears in PCT Publication WO 93/23028. In one embodiment the dipalmitoyl hydroxyproline is the triethanolamine salt of dipalmitoyl hydroxyproline.

8. Salicylic Acid Compound—The composition of the present invention may comprise a salicylic acid compound, and esters, salts, and derivatives thereof. In the compositions of the present invention, the salicylic acid compound may comprise from about 0.0001% to about 25%, alternatively from about 0.001% to about 15 alternatively from about 0.01% to about 10%, alternatively from about 0.1% to about 5%, and alternatively from about 0.2% to about 2%, of salicylic acid.

9. N-acyl Amino Acid Compound—The composition of the present invention may comprise one or more N-acyl amino acid compounds. In one embodiment, the N-acyl amino acid compound is selected from the group consisting of N-acyl Phenylalanine, N-acyl Tyrosine, their isomers, their salts, and derivatives thereof. The amino acid can be the D or L isomer or a mixture thereof.

Particularly useful as a topical skin tone evening (lightening or pigmentation reduction) cosmetic agent is N-undecylenoyl-L-phenylalanine, commercially available under the tradename Sepiwhite® from SEPPIC. This agent belongs to the broad class of N-acyl Phenylalanine derivatives, with its acyl group being a C11 mono-unsaturated fatty acid moiety and the amino acid being the L-isomer of phenylalanine.

The composition of the present invention may comprise from about 0.0001% to about 25%, alternatively from about 0.001% to about 10%, alternatively from about 0.01% to about 5%, and alternatively from about 0.02% to about 2.5% of the N-acyl amino acid

10. Sunscreen Actives—The composition of the present invention may comprise one or more sunscreen actives (or sunscreen agents) and/or ultraviolet light absorbers, and may be organic or inorganic. Examples of suitable sunscreen actives and ultraviolet light absorbers are disclosed in The Cosmetic, Toiletry, and Fragrance Association's The International Cosmetic Ingredient Dictionary and Handbook, 10th Ed., Gottschalck, T. E. and McEwen, Jr., Eds. (2004), p. 2267 and pp. 2292-93. Particularly suitable sunscreen actives include benzophenone, benzophenone-1, benzophenone-2, benzophenone-3, benzophenone-4, benzophenone-5, benzophenone-6, benzophenone-7, benzophenone-8, benzophenone-9, benzophenone-10, benzophenone-11, benzophenone-12, benzotriazolyl dodecyl p-cresol, 3-benzylidene camphor, benzylidene camphor sulfonic acid, benzyl salicylate, bis-ethylhexyloxyphenol methoxyphenyl triazine, bornelone, bumetrizole, butyl methoxydibenzoyl-methane, butyl PABA (p-aminobenzoic acid), cinnamidopropyl-trimonium chloride, cinoxate, dea-methoxycinnamate, dibenzoxazoyl naphthalene, di-t-butyl hydroxy-benzylidene camphor, diethylamino hydroxy-benzoyl hexyl benzoate, diethylhexyl butamido triazone, diethylhexyl 2,6-naphthalate, diisopropyl ethyl cinnamate, diisopropyl methyl cinnamate, di-methoxycinnamido-propyl ethyldimonium chloride ether, dimethyl PABA ethyl cetearyldimonium tosylate, dimorpholino-pyridazinone, dimorpholino-pyridazinone, disodium bisethylphenyl triaminotriazine stilbenedisulfonate, disodium distyrylbiphenyl disulfonate, disodium phenyl dibenzimidazole tetrasulfonate, drometrizole, drometrizole trisiloxane, ethyl dihydroxypropyl PABA, ethyl diisopropyl-cinnamate, ethylhexyl bis-isopentylbenzoxazolylphenyl melamine, ethyl dimethoxybenz-ylidene dioxoimidazolidine propionate, ethylhexyl dimethyl PABA, ethylhexyl methoxy-cinnamate, ethylhexyl methoxydibenzoyl-methane, ethylhexyl salicylate, ethylhexyl triazone, ethyl methoxycinnamate, ethyl PABA, ethyl urocanate, etocrylene, 4-(2-beta-glucopyrano-siloxy)propoxy-2-hydroxybenzophenone, glyceryl ethylhexanoate dimethoxycinnamate, glyceryl PABA, glycol salicylate, hexanediol disalicylate, homosalate, isoamyl cinnamate, isoamyl p-methoxycinnamate, isopentyl trimethoxy-cinnamate trisiloxane, isopropylbenzyl salicylate, isopropyl dibenzoylmethane, isopropyl methoxy-cinnamate, kaempferia galanga root extract, menthyl anthranilate, menthyl salicylate, methoxycinnamido-propyl hydroxysultaine, methoxycinnamido-propyl laurdimonium tosylate, 4-methylbenzylidene camphor, methylene bis-benzotriazolyl tetramethylbutyl-phenol, octocrylene, octrizole, PABA, PEG-25 PABA, phenylbenzimidazole sulfonic acid, polyacrylamidomethyl benzylidene camphor, polyamide-2, polyquaternium-59, polysilicone-15, potassium methoxy-cinnamate, potassium phenyl-benzimidazole sulfonate, red petrolatum, sodium benzotriazoyl butylphenol sulfonate, sodium phenylbenz-imidazole sulfonate, sodium urocanate, TEA-phenylbenzimidazole sulfonate, TEA-salicylate, terephthalylidene dicamphor sulfonic acid, tetrabutyl phenyl hydroxybenzoate, titanium dioxide, urocanic acid, zinc cerium oxide, zinc oxide, and mixtures thereof.

In one embodiment, the composition may comprise from about 1% to about 30%, and alternatively from about 2% to about 20%, of the sunscreen active and/or ultraviolet light absorber. Exact amounts will vary depending upon the chosen sunscreen active and/or ultraviolet light absorber and the desired Sun Protection Factor (SPF) and spectrum of protection (e.g. UV-A and/or UV-B), and are within the knowledge and judgment of one of skill in the art.

11. Water-Soluble Vitamins—The compositions of the present invention may comprise one or more water-soluble vitamins. Examples of water-soluble vitamins include, but are not limited to, water-soluble versions of vitamin B (such as vitamin B5 and vitamin B6), vitamin B derivatives, vitamin C (such as ascorbyl glucoside), vitamin C derivatives (such as magnesium ascorbyl phosphate, sodium ascorbyl phosphate, and ascorbyl palmitate), vitamin K, vitamin K derivatives, pro-vitamins thereof, such as panthenol and mixtures thereof. The composition may comprise from about 0.0001% to about 50%, alternatively from about 0.001% to about 10%, alternatively from about 0.01% to about 8%, and alternatively from about 0.1% to about 5%, of the vitamin compound.

12. Oil-Soluble Vitamins—The composition of the present invention may comprise one or more oil-soluble vitamins. Examples of oil-soluble vitamins include, but are not limited to, oil-soluble versions of vitamin D, vitamin D derivatives, vitamin E (such as vitamin E acetate), vitamin E derivatives, pro-vitamins thereof, and mixtures thereof. The composition may comprise from about 0.0001% to about 50%, alternatively from about 0.001% to about 10%, alternatively from about 0.01% to about 8%, and alternatively from about 0.1% to about 5%, of the oil-soluble vitamin compound.

13. Other actives—The compositions of the present invention may comprise one or more of the following other actives or ingredients: fatty acids (especially poly-unsaturated fatty acids), glucosamine, zinc pyrithione (ZPT), anti-fungal agents, thiol compounds (e.g., N-acetyl cysteine, glutathione, thioglycolate), other vitamins, beta-carotene, ubiquinone, idebenone, amino acids, hyaluronic acid increasing compounds/hyaluronidase inhibitors, indole-3-acetic acid, 6-benzylaminopurine, alpha-napthaleneacetic acid, 6-2-furoylaminopurine, Pluronic L43™, Tetronic 908™, tetronic 1107, green tea extract, white tea extract, menthol, tea tree oil, ginsenoside-RB1, ginsenoside-RB3, ginsenoside-RC, ginsenoside-RD, ginsenoside-RE, ginsenoside-RG1, ginseng root extract, ginseng flower extract, pomegranate extract, extracts from Ajuga turkestanica, and extracts from viola tricolor.

III. Dermatologically Acceptable Carrier

The personal care compositions of the present invention may comprise a dermatologically acceptable carrier. The dermatologically acceptable carrier may be present in an amount of from about 50% to about 99.99%, alternatively from about 60% to about 99.95%, alternatively from about 70% to about 98%, and alternatively from about 80% to about 95% by weight of the composition.

The carrier can be in a wide variety of forms. Non-limiting examples of emulsions useful herein include oil-in-water, water-in-oil, water-in-silicone, silicone in-water, water-in-oil-in-water, and oil-in-water-in-silicone emulsions. Alternatively, the emulsion is an oil-in-water emulsion. Emulsions also may contain a humectant, such as glycerin, and may contain from about 1% to about 10%, and alternatively from about 2% to about 5%, of a nonionic, anionic or cationic emulsifier. Examples of water-in-silicone and oil-in-water emulsions are described in U.S. Pat. No. 6,238,678, issued to Oblong et al.

Suitable emulsions may have a wide range of viscosities, depending on the desired product form. Exemplary low viscosity emulsions, which are preferred, have a viscosity of about 50 centistokes or less, more preferably about 10 centistokes or less, even more preferably about 5 centistokes or less.

The compositions of the present invention may also comprise other dermatologically acceptable topical carriers and can also comprise oral carriers. For example, another topical carrier can be a surfactant-containing cleanser (e.g., bar, shampoo, foaming cleanser, liquid cleanser, body wash, cleansing cloth, and the like). In such a carrier, the surfactant can be anionic, cationic, zwitterionic, nonionic, or mixtures of these. Another topical carrier example is a color cosmetic (lipstick, rouge, eye liner, mascara, foundation, nail polish, and the like). An oral carrier can be a beverage, food item, pill, capsule, powder, caplet, and the like.

V. Composition Forms

The personal care compositions of the present invention may be in a variety of forms, including but not limited to lotions, milks, mousses, serums, sprays, aerosols, foams, sticks, pencils, gels, creams and ointments, in-shower body lotions and/or body washes.

Compositions of this invention useful for cleansing (“cleansers”) may be formulated with a suitable carrier (e.g., as described above, and from about 1% to about 90%, by weight of the composition, of a dermatologically acceptable surfactant).

The compositions of the present invention may also be in the form of cosmetics. Suitable cosmetic forms include, but are not limited to, foundations, lipsticks, rouges, mascaras, and the like. Such cosmetic products may include conventional ingredients such as oils, colorants, pigments, emollients, fragrances, waxes, stabilizers, and the like. Exemplary carriers and such other ingredients which are suitable for use herein are described, for example, in U.S. Pat. No. 6,060,547.

VI. Methods for Regulating Mammalian Keratinous Tissue

The compositions of the present invention are useful for regulating the condition of and/or beautifying mammalian keratinous tissue conditions by stimulating aquaporin membrane proteins. The present invention provides for a method for regulating the condition of mammalian skin. Regulating skin condition means improving skin appearance and/or feel, for example, providing a smoother, more even appearance and/or feel, as described further herein.

The method of regulating skin condition comprises the step of topically applying to the skin and/or other keratinous tissue an effective amount of a personal care composition of the present invention. Alternatively, the method of regulating skin condition comprises the step of applying a composition as described herein to mammalian skin which exhibits signs of skin aging and/or one or more conditions described herein that may be regulated and/or improved. The amount of the composition applied, the frequency of application and the period of use will vary widely depending upon the level of components of a given composition and the level of regulation desired. For example, from about 0.01 g composition/cm² to about 1 g composition/cm² of keratinous tissue may be applied. In one embodiment, the compositions are applied at least once daily, where “daily” and “days” mean a 24-hour period. For example, the compositions may be applied daily for 30 consecutive days, alternatively for 14 consecutive days, alternatively for 7 consecutive days and alternatively for 2 consecutive days.

The application of the present compositions may occur through a variety of means, non-limiting examples of which include using the palms of the hands and/or fingers, in combination with a delivery enhancement device, a temperature-change element, a substrate, an implement (e.g., a cotton ball, swab, pad, substrate, etc.), and combinations thereof. The method may comprise the step of inducing a temperature change in the composition either simultaneously or sequentially with the step of applying the composition to the keratinous tissue. Alternatively, the method may comprise the step of inducing a temperature change in the keratinous tissue either simultaneously or sequentially with application of the composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) a member selected from a group consisting of kinetin, zeatin, and combinations thereof;
b) niacinamide; and
c) a dermatologically-acceptable carrier.

2. The composition of claim 1 wherein the composition comprises from about 0.05% to about 5%, by weight of the entire composition, of kinetin, zeatin, and combinations thereof.

3. The composition of claim 1 wherein the composition further comprises at least one compound selected from a group consisting of green tea extract; white tea extract;
menthol; tea tree oil; ginsenoside-RB1; ginsenoside-RB3; ginsenoside-RC; ginsenoside-RD; ginsenoside-RE; ginsenoside-RG1; ginseng root extract; ginseng flower extract; pomegranate extract, extracts from Ajuga turkestanica; extracts from viola tricolor; and combinations thereof

4. The composition of claim 1 wherein the composition further comprises at least one additional skin care active.

5. The composition of claim 5 wherein the additional skin care active is selected from a group consisting of sugar amines, retinoids, peptides, phytosterol, hexamidine, dialkanoyl hydroxyproline, salicylic acid, N-acyl amino acids, sunscreen actives, water soluble vitamins, oil-soluble vitamins, their derivatives, their precursors, and combinations thereof.

6. The composition of claim 6 wherein the composition further comprises deyhydroacetic acid, deyhydroacetic acid isomers, deyhydroacetic acid salts, or derivatives of deyhydroacetic acid.

7. The composition of claim 4 wherein the additional skin care active is hexamidine.

8. The composition of claim 4 wherein the additional skin care active is a sugar amine.

9. The composition of claim 8 wherein the sugar amine is N-acetyl glucosamine.

10. The composition of claim 4 wherein the additional skin care active is a peptide.

11. The composition of claim 10 wherein the peptide is palmitoyl-lysine-threonine, palmitoyl-lysine-threonine-threonine-lysine-serine, or combinations thereof.

12. The composition of claim 4 wherein the additional skin care active is an N-acyl amino acid.

13. The composition of claim 12 wherein the N-acyl amino acid is an N-acyl phenylalanine.

14. The composition of claim 1 further comprising from about 9% to about 30% of glycerin.

15. The composition of claim 1 comprising from about 1% to about 8% of niacinamide.

16. A method of regulating the condition of mammalian keratinous tissue comprising the step of applying to a portion of mammalian keratinous tissue in need of regulation a composition comprising:
a) from about 0.05% to about 5%, by weight, of kinetin, zeatin, and combinations thereof;
b) from about 1% to about 8%, by weight, of niacinamide; and
c) a dermatologically-acceptable carrier.

17. The method of claim 16 wherein the composition further comprises at least one additional skin care active.

18. The method of claim 17 wherein the additional skin care active is selected from a group consisting of sugar amines, retinoids, peptides, phytosterol, hexamidine, dialkanoyl hydroxyproline, salicylic acid, N-acyl amino acids, sunscreen actives, water soluble vitamins, oil-soluble vitamins, their derivatives, their precursors, and combinations thereof.

19. A personal care composition comprising:
a) an N-acyl amino acid compound selected from the group consisting of N-acyl amino acid, its isomers, its salts, and derivatives thereof;
b) a skin care active selected from the group consisting of sugar amine, vitamin B₃, retinoids, peptides, phytosterol, dialkanoyl hydroxyproline, hexamidine, salicylic acid and their derivatives, and combinations thereof;
c) a member selected from a group consisting of kinetin, zeatin, and combinations thereof; and
d) a dermatologically-acceptable carrier.

20. The personal care composition of claim 19 wherein the N-acyl amino acid is an N-acyl phenylalanine.

21. The personal care composition of claim 19 wherein the composition further comprises deyhydroacetic acid, deyhydroacetic acid isomers, deyhydroacetic acid salts, or derivatives of deyhydroacetic acid.

22. The personal care composition of claim 19 wherein said composition comprises
from about 0.0001% to about 25%, by weight, of said N-acyl amino acid compound selected from the group consisting of N-acyl amino acid, its isomers, its salts, and derivatives thereof; and
from about 0.001% to about 10%, by weight, of said member selected from a group consisting of kinetin, zeatin, and combinations thereof.

23. The method of regulating the condition of mammalian keratinous tissue comprising the step of applying to a portion of mammalian keratinous tissue in need of regulation the composition of claim 19.

24. A personal care composition, comprising:
a) a hexamidine compound selected from the group consisting of hexamidine, its salts, and its derivatives;
b) a skin care active selected from the group consisting of sugar amines, vitamin B₃, retinoids, peptides, phytosterols, their derivatives, and combinations thereof;
c) a member selected from a group consisting of kinetin, zeatin, and combinations thereof; and
d) a dermatologically acceptable carrier.

25. The personal care composition of claim 24 wherein said composition comprises
from about 0.0001% to about 25%, by weight, of said hexamidine compound selected from the group consisting of hexamidine, its salts, and its derivatives; and
from about 0.001% to about 10%, by weight, of said member selected from a group consisting of kinetin, zeatin, and combinations thereof.

26. The personal care composition of claim 24 wherein the composition further comprises deyhydroacetic acid, deyhydroacetic acid isomers, deyhydroacetic acid salts, or derivatives of deyhydroacetic acid.

27. The method of regulating the condition of mammalian keratinous tissue comprising the step of applying to a portion of mammalian keratinous tissue in need of regulation the composition of claim 24.
